(19) United States
(12) Patent Application Publication
(10) Pub. No.: US 2004/0071588 A1
(43) Pub. Date: Apr. 15, 2004

(54) Method of disinfecting, freshening and perfuming the air based on essential oils and/or the active ingredients thereof

(76) Inventors: Jordi Basaganas Millan, Cerdanyola del Valles (ES); Antonio Ruiz Sanchez, Armilla (ES)

Correspondence Address: SCHIFF HARDIN, LLP, PATENT DEPARTMENT, 6600 SEARS TOWER, CHICAGO, IL, 60606-6473, US

(21) Appl. No.: 10/385,797

(22) Filed: Mar. 11, 2003

(30) Foreign Application Priority Data
Oct. 15, 2002 (WO) PCT/ES02/00486

(51) Int. Cl. A61L 9/00

(52) U.S. Cl. 422/4; 422/5; 422/305; 422/306; 422/1

(57) ABSTRACT

The invention provides a method for disinfection or the inhibition of microbial growth in the air, freshening and perfuming of the air, being based on the emission of particles into the air of a disinfecting composition based on essential oils and/or their active ingredients their mixtures with glycols and/or glycol ethers in variety of proportions. In accordance with the method of the invention, the particles emitted into the air which it is sought to disinfect, freshen and perfume, are produced using the evaporation of the liquid composition, through a wick indirectly heated to a temperature of less than 100° C. in its upper area.

### BACKGROUND OF THE INVENTION

1. Field of the Invention

The present invention relates to a method of disinfecting, freshening and perfuming the air based on the emission of particles into the air of a disinfecting and aromatic composition based on essential oils and/or their active ingredients, in which the particles are produced using evaporation through a wick indirectly heated in an upper area.

2. Description of The Related Art

It is well known that the air in an enclosed space is colonized by bacterial flora of a diverse nature. On numerous occasions, such enclosed spaces can remain closed for hours or have a deficient ventilation and renovation of air so that the existing bacterial accumulation can become infectious. A large number of microorganisms use air as a propagation medium, affecting individuals and residents in these spaces through their respiratory tract.

The present invention is directly related with international patent application PCT/ES 01/00443, filed on 20 Nov. 2001, and herein incorporated by reference. This application describes a method of disinfecting, freshening and perfuming the air based on essential oils and/or their active ingredients, which the method comprising producing an emission of particles into the air of a disinfecting, freshening and perfuming composition which comprises essential oils and/or their active ingredients, and in that the particles of the disinfecting composition are produced using evaporation through a wick indirectly heated in its upper area. The temperatures reached by this upper area of the wick are equal to or greater than 100° C.

Moreover, systems are known to feed insecticides into the air through producing the evaporation of an insecticide liquid through an absorbent wick immersed in the liquid itself so that by heating the wick, the active ingredient of the insecticide liquid evaporates into the atmosphere. U.S. Pat. No. 4,745,705 discloses a method of feeding insecticides into the air based on the above mentioned principle.

U.S. Pat. Nos. 5,095,647; 4,663,315, 5,038,394; and 5,290,546, describe systems for feeding insecticides into the air without describing the devices employed in the generation of particles.

Furthermore, British patent number GB A2 194442 discloses a fumigator device capable of transpiring a chemical agent of insecticidal, disinfecting, deodorant, etc., action through a porous wick and indirect heating for the purpose of killing insects, germs or deodorizing, although this patent does not describe explicitly the method of disinfection nor the compositions for the application of the corresponding chemical agent.

U.S. Pat. No. 5,591,395 discloses a method for disinfecting the air using of particle generators for the release of disinfecting agents into the atmosphere, so that in this patent 90% of the particles emitted have a size limited to 5 microns, being likewise equally limited the range of working temperature, between 50 and 120° C.

Other documents and patents are known that concern the disinfection of surfaces through liquid formulations or aerosol generators, although the products are not disclosed for disinfecting of air, and in such sense mention can be made of the methods and/or formulations of disinfecting action which employ essential oils as active ingredients, disclosed in the Spanish Patents ES 2023530 and ES 2143172, as well as in EP 842604 A1 and WO96/39826.

### SUMMARY OF THE INVENTION

The main aspect of the present invention comprises a method of disinfecting, freshening and perfuming the air based on essential oils and/or their active ingredients which consists in producing through evaporation, using an indirectly heated wick, an emission of particles into the air of a disinfecting, freshening, and perfuming composition which comprises essential oils and/or their active ingredients, having a wick that reaches a temperature of less than 100° C. in its upper area.

### DESCRIPTION OF THE DRAWING

FIG. 1 is an isometric view of an embodiment of an inventive device configured to disinfect, freshen and perfume the air.

### DETAILED DESCRIPTION OF THE PREFERRED EMBODIMENTS

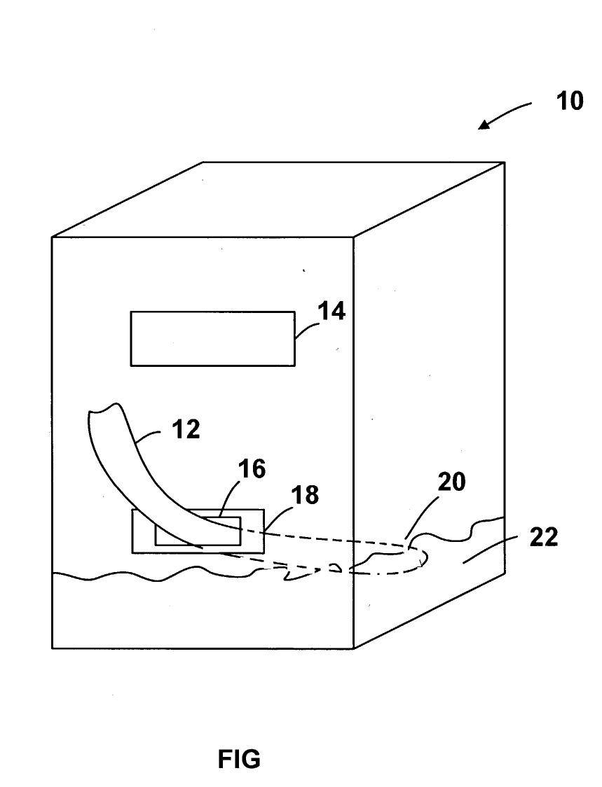

FIG. 1 illustrates an embodiment of the inventive device 10, which comprises a container that comes with an opening in its upper part 14 and another in the lower part 16, the lower part opening 16 having a porous or absorbent wick 12 to conduct a liquid 22 from the container to the exterior through the lower opening 16. The lower portion 20 of the wick 12 is in contact with the liquid 22; surrounding this opening 16 of the container is a heater element 18 which is designed in such a way that it only reaches temperatures of less than 100° C. Any conventional mechanism for such temperature control may be used.

An embodiment of the inventive method is also provided. Using the method of the invention, a high mortality is achieved of certain microorganisms dispersed in the environmental air of a room, which can be pathogenic or transmit infections, or achieve the inhibition of microbial growth in the actual environmental air together with a freshening or perfuming of the air.

Preferably the disinfecting compositions also include glycols and/or glycol derivatives like, for example, glycol ethers in a variety of proportions.

The method of disinfecting, freshening and perfuming finds special application in the household and institutional field, for disinfecting, freshening and perfuming the air in sitting rooms, bedrooms or other enclosures or closed spaces, so that the use of essential oils and/or their active ingredients, as disinfecting and aromatic additives and/or their mixtures with glycols and/or glycol ethers in variety of proportions, offer the following advantage with regard to other conventional methods of disinfection.

The safety profile of essential oils and/or their active ingredients is much superior to that of the majority of non-natural antimicrobial additives. In fact, the majority of essential oils of interest are accepted as aromatic agents, natural repellants, food additives-or natural preservatives for human or animal consumption.

The essential oils and/or their active ingredients possess intrinsic odourous properties, where they provide or enhance the smell of the disinfecting product, allowing the composition to have freshening or aromatic properties without the necessity to add an additional perfume.

In the context of the present invention essential oil is understood to be those volatile organic substances of botanical origin constituents of plants, trees and shrubs. Essential oils can be obtained from cinnamon, the tea tree, citronella, lemongrass, thyme, citrus, anisette, cloves, geranium, rose, mint, lavender, eucalyptus, peppermint, camphor, sandalwood, cedarwood and their mixtures, without essential oils being limited to those obtained from the previously mentioned products.

For its part, within the context of the present invention, the active ingredient of essential oils is understood to be those chemical compounds which have shown a proven activity in the field of disinfection. The active ingredients of essential oils are preferably terpineol, cinnamaldehyde, cinnamic acid, thymol, citronellal, eugenol, menthol, geraniol, eucalyptol (cineol), cedrol, anethol, cajeputene, carbacrol and their mixtures, without the active agents of interest being limited to these ingredients.

In a preferred manner, the essential oils for use in the present invention, are the oils of the tea tree, cinnamon, citronella, artemisia, lemongrass, cedar, cloves, pine, bergamot and lavandino, whilst the main active agents are thymol, citronellal, citronellol, estragol, geranyl acetate, eucalyptol, eugenol, terpineol, cinnamaldehyde, cinnamic acid, citral, dihydromyrcenol, rose oxide and cajeputene.

The glycols employed as solvents for these essential oils and/or their active agents can preferably be propylene glycol, dipropylene glycol, triethylene glycol, glycol ethers and their mixtures.

Although the effectiveness is known of the glycols as air disinfecting agents, as is mentioned in the U.S. Pat. No. 5,591,395, the use of essential oils in the process of air disinfection and their mixtures with glycols offers a clear advantage over the use of only glycols as disinfecting agents. In this sense, there are numerous bibliographical studies which demonstrate that the disinfecting action of glycols is highly dependent upon ambient conditions of temperature and relative humidity. Thus, humidities greater than 35-40% reduce and can even suppress their disinfecting effectiveness. This is due to the good hygroscopic properties of glycols, which produces their condensation, minimizing the effective amount of agent in the vapour state in the air.

These bibliographical studies, herein incorporated by reference, correspond to:

Jennings, B. H. et al. (1944) The uses of glycol vapors for air sterilization and control of airborne infection;

Robertson, O. H. (1943) Sterilization of air with glycol vapors;

Lester, William et al. (1949) The rate of bactericidal action of triethylene glycol vapor on microorganisms dispersed into the air in small droplets;

Hamburger, Morton et al. (1945) The present status of glycol vapors in air sterilization;

Bigg, Edward et al. (1945) Epidemiologic observations on the uses of glycol vapors for air sterilization; and

Puck, Theodore T. et al. (1943) The bactericidal action of propylene glycol vapor on microorganisms in air.

The disinfecting effectiveness of essential oils and/or their active ingredients or their mixtures with glycols are not dependent on the conditions of temperature and relative humidity, since these agents have scant or no hygroscopy, remaining therefore in the vapour phase and as active agents for disinfecting the air.

In a particular embodiment of the invention, the disinfecting composition, besides essential oils and/or their active ingredients, as well as glycols, includes aromatic compounds which do not affect the disinfecting activity of the product and their function is solely that of contributing a more pleasant and appropriate odor to the products. In the context of the present invention, aromatic compounds are understood to be those substances which have odorising properties. The aromatic compounds employed in the present invention include, although they are not limited to, chemical compounds like aldehydes (phenylacetic, C8, C9, C10, etc.), acetates (of benzyl, hexyl, octyl, etc.), ionones (gamma-methyl, beta, etc.), lactones (gamma undecalactone, decalactone, nonalactone, dodecalactone, etc.), alcohols (phenylethyl, phenylpropyl), pyrazines, or acids.

The emission of the disinfecting and aromatic particles into the air will be carried out through the evaporation of the disinfecting and aromatic liquid using indirect heating of the upper part of a porous wick immersed in the liquid, the dispersion system possibly comprising an external cell or container with an opening in its upper part, the cell containing the disinfecting and aromatic liquid in its interior and will allow the collocation of a porous or absorbent wick to conduct the liquid to the exterior, so that surrounding the opening of the cell is a heater element, which is limited in order not to reach temperatures of 100° C. or higher, and which can be one of the conventional heater elements, such as heater rings or one or several PTCs.

In any case, the heater element will be connected using a conductor to a source of electric current, which can be a battery, a cell, or a household connection to the electricity mains.

The absorbent wick can be made of any conventional material employed for such a purpose. These materials could include ceramic materials, or materials based on polyester or polyethylene fibers, compacted wood, sintered plastics and even carbon fibers.

According to the present invention the wick is heated indirectly to a temperature of less than 100° C. in its upper area. Working at these temperatures, a very effective and satisfactory operation is achieved of the method of disinfecting, freshening and perfuming; moreover, this avoids the total or partial degradation of some essential oils and/or their active ingredients. Thus, as well as enhancing the effectiveness of the method of desinfectating, freshening and perfuming, the variety of essential oils and/or active ingredients that can be used in the method of the invention is more extensive than in the conventional methods.

Evidently, the effectiveness of disinfecting the air is determined by evaluation of the decrease in the microbial pollution with respect to the initial situation after having used the disinfecting product.

Experiments have been carried out using different types of formulation of the essential oil and/or its active ingredients, the references made, among others, being summarized in the following examples:

### EXAMPLE 1

The formulation tested in this case contains 40% of active ingredients, 10% of inactive aromatic ingredients and 50% of glycol ethers as solvents. Specifically, the active components which it contains are:

| COMPONENTS | % IN FORMULA |
|---|---|
| Caraway | 0.375 |
| Cedar | 0.15 |
| Cinnamon | 0.25 |
| Cypress | 0.275 |
| Dihydromyrcenol | 11 |
| Cloves | 4.9 |
| Dehydro methyl jasmonate | 4.25 |
| Orange | 9.75 |
| Patchouli | 1.1 |
| Sage | 1.1 |
| Mint | 0.55 |
| Absinthe | 0.05 |
| Armoise | 0.05 |
| Sandalwood | 1.1 |
| Geranium | 0.75 |
| Thyme | 3.25 |
| Verbena | 1.1 |
| TOTAL | 40.0 |

Procedure:

In a room of 1000 litres, between 0.12 and 0.18 grams of the disinfecting formulation are spread using evaporation through wicks of polyester fiber heated indirectly in its upper part with a PTC heater system of 75° C. working temperature. At that point in time, a controlled quantity of microbial pollution is introduced. A contact time of 30 minutes is allowed between the microorganism under study and the active ingredients dispersed in the air. On completion of that half hour, an air capture is carried out and the corresponding count is made of the number of colonies. This quantity corresponds to the microbial load in the air after the disinfecting product has acted on the initial pollution for a half hour.

The results are the following:

| MICROORGANISM | % MORTALITY |
|---|---|
| Salmonella cholerae | 100% |
| Microccocus luteus | 94% |
| Aspergillus Fumigatus | 96% |

These results reflect again an important reduction in the quantity of microorganisms present in the air, demonstrating the high disinfecting power of the formula.

### EXAMPLE 2

The formula tested in this case contains 37.5% of active ingredients, 12.5% of inactive aromatic ingredients, and 50% of glycol ethers as solvents. Specifically, the active components that it contains are:

| COMPONENTS | % IN FORMULA |
|---|---|
| Etemi | 0.5 |
| Anisette | 0.5 |
| Eucalyptus | 0.7 |
| Lavender | 6.2 |
| Petitgrain | 0.4 |
| Pine | 5 |
| Basil | 0.25 |
| Sage | 0.2 |
| Rose | 0.2 |
| Orange | 1.5 |
| Bergamot | 2.5 |
| Lime | 2 |
| Cinnamon | 0.2 |
| Cloves | 0.25 |
| Cedar | 4 |
| Lemon | 6.2 |
| Geranium | 6.2 |
| Litsea Cubeba | 0.4 |
| Mint | 0.1 |
| Thyme | 0.2 |
| TOTAL | 37.50 |

Procedure:

The same procedure as for Example 1; the results are the following:

| MICROORGANISM | % MORTALITY |
|---|---|
| Salmonella cholerae | 100% |
| Microccocus luteus | 99% |
| Aspergillus Fumigatus | 99% |

These results reflect again an important reduction in the quantity of microorganisms present in the air, showing the high disinfecting power of the formula.

### EXAMPLE 3

The formula tested in this case contains 35.0% of active ingredients, 15% of inactive aromatic ingredients, and 50% of glycol ethers as solvents. Specifically, the active components that it contains are:

| COMPONENTS | % IN FORMULA |
|---|---|
| Pine | 15 |
| Eucalyptus | 2.5 |
| Orange | 2.5 |
| Lime | 1 |
| Sage | 1 |
| Lavandin | 0.75 |
| Fir | 1 |
| Rosemary | 0.25 |
| Juniper berry | 1 |
| Linalol | 6 |
| Petit rain | 1.5 |
| Caraway | 1 |
| Cedar | 1 |
| Bergamot | 0.25 |
| Nutmeg | 0.25 |
| TOTAL | 35.0 |

Procedure:

The same procedure as for Example 1; the results are the following:

| MICROORGANISM | % MORTALITY |
|---|---|
| Salmonella cholerae | 100% |
| Microccocus luteus | 96% |
| Aspergillus Fumigatus | 99% |

These results reflect again an important reduction in the quantity of microorganisms present in the air, showing the high disinfecting power of the formula.

The inactive aromatic ingredients do not affect the disinfecting activity of the product and their function is solely that of contributing a more pleasant and more appropriate odor to the products. This is demonstrated with the study of the same formulas with active ingredients and without aromatic components, as is detailed in the following examples:

### EXAMPLE 4

The formula tested in this case contains 45% of active ingredients and 55% of glycol ethers as solvents. Specifically, the active components which it contains are the same ones and in approximately equal proportion to that in example 1:

| COMPONENTS | % IN FORMULA |
|---|---|
| Caraway | 0.4 |
| Cedar | 0.25 |
| Cinnamon | 0.35 |
| Cypress | 0.3 |
| Dihydromyrcenol | 11.5 |
| Cloves | 5.5 |
| Dehydro methyl jasmonate | 4.5 |
| Orange | 10 |
| Patchouli | 1.5 |
| Sage | 1.5 |
| Mint | 1.0 |
| Absinthe | 0.1 |
| Armoise | 0.1 |
| Sandalwood | 1.5 |
| Geranium | 1.25 |
| Thyme | 3.75 |
| Verbena | 1.5 |
| TOTAL | 45.0 |

Procedure:

The same as that in Example 1; the results are the following:

| MICROORGANISM | % MORTALITY |
|---|---|
| Salmonella cholerae | 99% |
| Microccocus luteus | 95% |
| Aspergillus Fumigatus | 95% |

These results reflect again an important reduction in the quantity of microorganisms present in the air, showing the high disinfecting power of the formula in the absence of inactive aromatic agents.

### EXAMPLE 5

The formula tested in this case contains 42% of active ingredients and 58% of glycol ethers as solvents. Specifically, the active components that it contains are the same ones in approximately equal proportion to that in example 2:

| COMPONENTS | % IN FORMULA |
|---|---|
| Elemi | 0.55 |
| Anisette | 0.55 |
| Eucalyptus | 0.7 |
| Lavender | 6.2 |
| Petitgrain | 0.4 |
| Pine | 6 |
| Basil | 0.25 |
| Sage | 0.2 |
| Rose | 0.2 |
| Orange | 1.5 |
| Bergamot | 2.5 |
| Lime | 2 |
| Cinnamon | 0.2 |
| Cloves | 0.25 |
| Cedar | 5 |
| Lemon | 7.2 |
| Geranium | 7.2 |
| Litsea Cubeba | 0.4 |
| Mint | 0.1 |
| Thyme | 0.2 |
| TOTAL | 42.0 |

Procedure:

The same protocol as for Example 1; the results are the following:

| MICROORGANISM | % MORTALITY |
|---|---|
| Salmonella cholerae | 100% |
| Microccocus luteus | 99% |
| Aspergillus Fumigatus | 99% |

These results reflect again an important reduction in the quantity of microorganisms present in the air, showing the high disinfecting power of the formula in the absence of non-active aromatic agents.

### EXAMPLE 6

The formula tested in this case contains 44% of active ingredients and 56% of glycol ethers as solvents. Specifically, the active components that it contains are the same ones and in approximately equal proportion to that in Example 3:

| COMPONENT | % IN FORMULA |
|---|---|
| Pine | 17 |
| Eucalyptus | 3 |
| Orange | 3 |
| Lime | 1.5 |
| Sage | 1.5 |
| Lavandin | 1.75 |
| Fir | 1.5 |
| Rosemary | 0.75 |
| Juniper berries | 1.5 |
| Linalol | 7 |
| Petitgrain | 1.5 |
| Caraway | 1.5 |
| Cedar | 1.5 |
| Bergamot | 0.5 |
| Nutmeg | 0.5 |
| TOTAL | 44.0 |

Procedure:

The same protocol as for Example 1; the results are the following:

| MICROORGANISM | % MORTALITY |
|---|---|
| Salmonella cholerae | 100% |
| Microccocus luteus | 97% |
| Aspergillus Fumigatus | 99% |

These results reflect again an important reduction in the quantity of microorganisms present in the air, showing the high disinfecting power of the formula in the absence of inactive aromatic agents.

For the purposes of promoting an understanding of the principles of the invention, reference has been made to the preferred embodiments, and specific language has been used to describe these embodiments. However, no limitation of the scope of the invention is intended by this specific language, and the invention should be construed to encompass all embodiments that would normally occur to one of ordinary skill in the art.

The particular implementations shown and described herein are illustrative examples of the invention and are not intended to otherwise limit the scope of the invention in any way. For the sake of brevity, conventional knowledge may not be described in detail. Moreover, no item or component is essential to the practice of the invention unless the element is specifically described as “essential” or “critical”. Numerous modifications and adaptations will be readily apparent to those skilled in this art without departing from the spirit and scope of the present invention.

1. A method of disinfecting, freshening and perfuming the air, comprising:
providing at least one of essential oils and their active ingredients to a container;
providing an indirectly heated wick having an upper area; and
indirectly heating the upper area of the wick to a temperature of less than 100° C. during times of normal operation, thereby causing an emission of particles into the air of a disinfecting, freshening and perfuming composition which comprises at least one of essential oils and their active ingredients.

2. The method according to claim 1, wherein the disinfecting composition comprises at least one of glycols and derivatives of glycols as a dissolving medium of the essential oils and their active ingredients.

3. The method according to claim 1, further comprising:
obtaining essential oils from at least one of a tea tree, leaves of Ceylon cinnamon, citronella, artemisia, lemongrass, cedar, cloves, pine, bergamot, lavandino and their mixtures; and
providing at least one of thymol, citronellal, citronellol, estragol, geranyl acetate, eucalyptol, eugenol, terpineol, cinnamaldehyde, cinnamic acid, citral, dihydromyrcenol, rose oxide, cajeputene and their mixtures as the active ingredients.

4. The method according to claim 2, further comprising:
providing glycols that are selected from among the group consisting of propylene glycol, dipropylene glycol, triethylene glycol, glycol ethers and their mixtures.

5. The method according to claim 1, further comprising:
providing aromatic compounds to the disinfecting composition.

6. A device for disinfecting, freshening and perfuming the air, comprising:
an opening in an upper part;
an opening in a lower part;
at least one of essential oils and their active ingredients located in an interior of the device;
a porous or absorbent wick configured to extend from a device exterior through the lower opening into the device interior and into the essential oils or their active ingredients and configured to conduct these materials from the interior to the exterior of the container; and
a heater element configured to reach temperatures less than 100° C. during normal operation, the heater element surrounding the lower opening.

7. A method for using a mixture for disinfecting, freshening, perfuming the air, comprising:
providing at least one of essential oils and their active ingredients for the mixture;
providing at least one of glycols and derivatives of glycols for the mixture;
providing aromatic compounds for the mixture;
providing an indirectly heated wick having an upper area; and
providing a heater configured to indirectly heat an upper area of the wick to a temperature of less than 100° C. during times of normal operation, thereby causing an emission of particles into the air of the mixture.
